(19) United States
(12) Patent Application Publication
(10) Pub. No.: US 2017/0215967 A1
(43) Pub. Date: Aug. 3, 2017

(54) IMPLANT RECOMMENDATION SYSTEM AND METHOD

(71) Applicant: William B. Spath, Costa Mesa, CA (US)

(72) Inventor: William B. Spath, Costa Mesa, CA (US)

(21) Appl. No.: 15/421,552

(22) Filed: Feb. 1, 2017

Related U.S. Application Data
(60) Provisional application No. 62/290,723, filed on Feb. 3, 2016.

(51) Int. Cl. A61B 34/10; G06F 19/00; A61F 2/32

(52) U.S. Cl. CPC A61B 34/10; A61F 2/32; G06F 19/322; G06F 19/321; A61B 2034/108; A61F 2002/30952

(57) ABSTRACT

Making a recommendation concerning a surgical procedure in a being is performed by a computer including a computer readable memory storing executable instructions and a processor in communication with the computer readable memory. The method includes a) acquiring information pertaining to the being and b) determining a recommendation pertaining to the being using the acquired information. In particular, the recommendation includes one of: a) one or more options for a new or replacement implant for inserting into the being; or b) a technique to be used for revision of an existing implant in the being. The method also includes transmitting, by the computer, the recommendation.

### CROSS REFERENCE TO RELATED APPLICATION

This application claims the benefit of U.S. Provisional Patent Application Ser. No. 62/290,723, filed Feb. 3, 2016, having attorney docket no. ZZZ 064 MA, and entitled “IMPLANT RECOMMENDATION AND WEAR RECOGNITION SYSTEM AND METHOD”, the entire disclosure of which is hereby incorporated by reference herein.

### FIELD OF THE INVENTION

The present invention relates to the field of medical implants and, more particularly to recommendation of medical implants.

### BACKGROUND OF THE INVENTION

In general, the revision rate for medical implants is growing at a very rapid rate and as the number of patients who have received implants increases, this revision rate will only continue to grow. Patient records may not include specific or accurate identifying information about objects that have been implanted secondary to poor record keeping, lost records or non-existent records due to international travel. The record may indicate that a device was implanted but may not include the specific model, lot, size, varying combinations or other information about the particular implant.

Currently, one method to identify an implant involves x-raying the implant and e-mailing that x-ray to one or more experienced manufacturer representatives and asking them to guess at the identity of the implant. Incorrect implant identification can potentially delay onset of surgery, prolong length of surgical time, increase surgical complexity, increase morbidity and/or mortality secondary to increased infections or prolonged anesthetic time, degrade the quality of post-operative mechanical or functional outcome, or increase the potential for additional revision surgery. All of the aforementioned complications will ultimately increase the cost of care.

### SUMMARY OF THE INVENTION

Aspects of the present invention relate to a computer-implemented method for making a recommendation concerning a surgical procedure in a being, the computer including a computer readable memory storing executable instructions and a processor in communication with the computer readable memory. The method comprises:

acquiring, by a computer, information pertaining to the being, determining, by the computer, a recommendation pertaining to the being using the acquired information, the recommendation comprising one of: one or more options for a new or replacement implant for inserting into the being; or a technique to be used for revision of the existing implant; and transmitting, by the computer, the recommendation.

Additional aspects of the present invention relate to a computer-implemented method for determining a change in characteristics of an implant in a being, the computer including a computer readable memory storing executable instructions and a processor in communication with the computer readable memory. The method comprises acquiring, by a computer, measurement information pertaining to the implant in the being, the measurement information comprising at least one of a size, shape, and definition of the implant; comparing, by the computer, the acquired measurement information with known virgin measurement information pertaining to the implant; determining, by the computer, comparison information based on the comparison between the acquired measurement information and the virgin measurement information; and transmitting, by the computer, the comparison information.

### BRIEF DESCRIPTION OF THE DRAWINGS

While the specification concludes with claims particularly pointing out and distinctly claiming the present invention, it is believed that the present invention will be better understood from the following description in conjunction with the accompanying Figures, in which like reference numerals identify like elements, and wherein:

FIG. 1A depicts a flowchart of an example method of identifying implants in accordance with the principles of the present invention;

FIG. 1B depicts a flowchart of an example method for refining a selection of matching implants in accordance with the principles of the present invention;

FIG. 2A depicts a flowchart of an example method for identifying matching implants in a database in accordance with the principles of the present invention;

FIGS. 2B-2D depict an example hip implant having example features of interest marked with circles;

FIG. 3 depicts a system for identifying implants in accordance with the principles of the present invention;

FIG. 4 is a block diagram of a data processing system in accordance with the principles of the present invention;

FIG. 5 is a flowchart of an example method for recommending an implant;

FIG. 6 is a flowchart of an example method for estimating wear of an implant;

FIG. 7 depicts a flowchart of an example method, from the perspective of an implant recommendation system, for providing a recommendation in accordance with the principles of the present invention; and

FIG. 8 depicts a block-level diagram of an implant recommendation system in accordance with the principles of the present invention.

### DETAILED DESCRIPTION OF THE INVENTION

In the following detailed description of the preferred embodiment, reference is made to the accompanying drawings that form a part hereof, and in which is shown by way of illustration, and not by way of limitation, a specific preferred embodiment in which the invention may be practiced. It is to be understood that other embodiments may be utilized and that changes may be made without departing from the spirit and scope of the present invention.

One possible methodology for identifying undocumented implants utilizing visual identification from images obtained from varying Radiology modalities may rely on experienced health care providers and/or device manufacturing representatives to guess at the identity of the implant. Incorrect implant identification can potentially delay onset of surgery, prolong length of surgical time, increase surgical complexity, increase morbidity and/or mortality secondary to increased infections or prolonged anesthetic time, degrade the quality of post-operative mechanical or functional outcome, or increase the potential for additional revision surgery. All of the aforementioned complications will ultimately increase the cost of care.

The term “internal medical image” is used herein to encompass any of a wide variety of techniques for capturing images of an implant inside of a patient. Various imaging or scanning technologies are contemplated that allow an image of the implant inside the patient to be acquired in such a way that the implant may be visually distinguished, in the image, from the body tissue in the vicinity of the implant. For example, an internal medical image may be captured using non-invasive techniques such as CT scanning, PET scanning, MRIs, other 3D imaging techniques, X-ray, other radiography techniques, ultrasound scanning, and multi-dimensional ultrasound techniques and invasive techniques involving endoscopy, digital videos or still pictures. Thus, two-dimensional images may be captured using some techniques and higher-dimensional images can be acquired using alternative techniques. Also, one of ordinary skill will recognize that one or more two-dimensional images of the implant can be constructed from volumetric data that may be acquired.

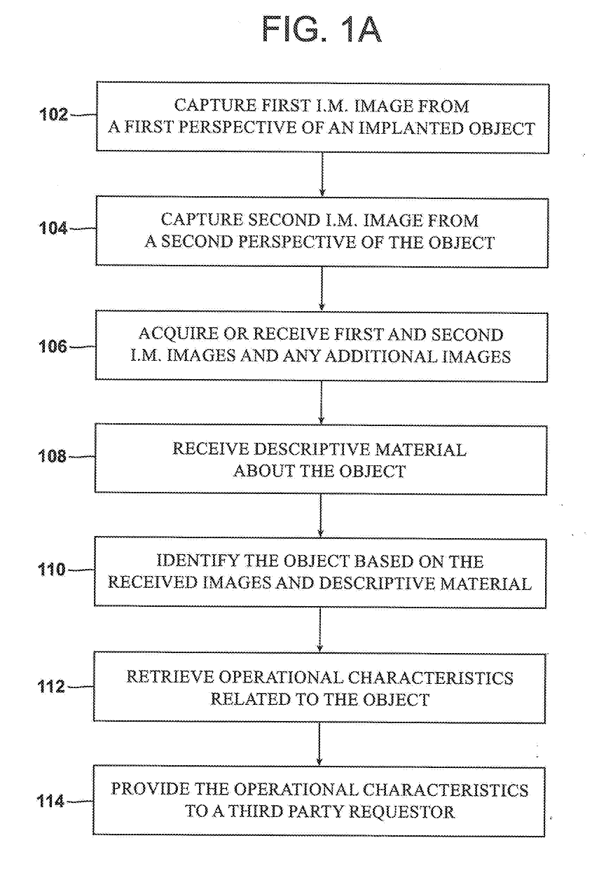

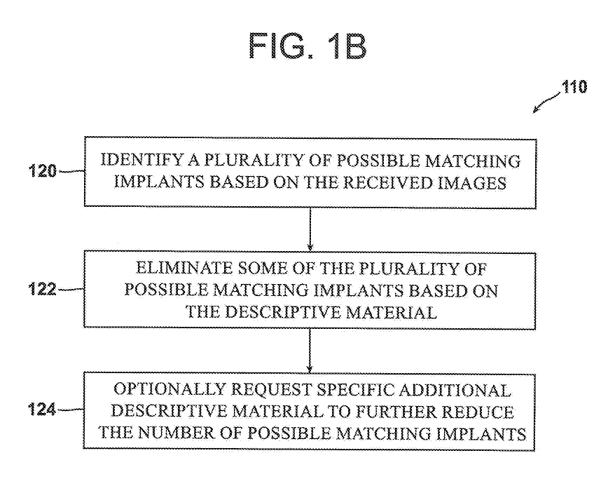

Additionally, some of the examples described herein may refer to particular perspectives used to acquire an internal medical image. These example perspectives are provided merely to help describe specific examples but one of ordinary skill will recognize that internal medical images may be acquired from a wide variety of different perspectives without departing from the scope of the present invention. However, some standardized poses or perspectives or positions may be beneficial according to certain aspects of the present invention. For example, as a minimum, a first internal medical image may be taken from a lateral perspective (i.e., a patient's side facing an x-ray source, for example) and a second internal medical image can be taken from one of a posterior-anterior (i.e., the patient's back facing the, for example, x-ray source) perspective or an anterior-posterior (i.e., the patient's front facing the, for example, x-ray source) perspective. Those two internal medical images would provide images of an implant from generally two orthogonal perspectives and may be used to identify many more attributes and features of an implant than merely a single two-dimensional image. FIG. 1A depicts a flowchart of an example method of identifying implants in accordance with the principles of the present invention. There may be a variety of instances in which it is beneficial to identify an object, such as a medical implant, that has been previously implanted in a patient, such as a person, other living being such as an animal, or a non-living being, such as a cadaver. Before performing surgery or diagnostic tests on a patient, it might be helpful to know what medical implants that patient may have as well as some operational characteristics about the implants. For example, if an implant has been subject to a recall or if an expected life of the implant has expired or if the patient's clinical status warrants intervention and the implant should be replaced or revised, it may be beneficial to know the identity and operational characteristics of any such implants thereby optimizing the surgical technique required to perform a surgical intervention.

There may also be legal or criminal reasons to identify medical implants that were implanted in a patient, even if that patient is no longer living. Thus, embodiments of the present invention are useful to medical personnel as well as non-medical personnel. However, the term “patient” is used herein to generally describe the living or non-living being into which an object, such as a medical-related implant, was implanted.

According to the example flowchart of FIG. 1A, at least two different internal medical images are captured in steps 102 and 104. In particular, each of the two internal medical images are captured from a different perspective (e.g., anterior-posterior, lateral, etc.) and each internal medical image relates to a vicinity around the object implanted in the patient. One of ordinary skill will recognize that a trained technician is typically involved with capturing an internal medical image and will know an appropriate method and region for capturing a useful internal medical image of the implanted object. It is noted that the two captured images could be derived from the same technology, e.g., they could both be x-rays, or they could be derived from different technologies, e.g., one could be an x-ray while the other is an MRI, etc.

Next, in step 106, the captured internal medical images, which may be two or more different images, are acquired by the operator of a system for identifying implants. The personnel that provide the internal medical images to the implant identification system can be located remotely from the implant identification system such as, for example, at a medical facility, a diagnostic lab, a police station, or a lawyer's office. These remotely located personnel will have copies, whether electronic or film, of the internal medical images of the implanted object and can provide them to the implant identification system in a myriad of ways without departing from the scope of the present invention.

In addition to the two internal medical images (or more), various three-dimensional, four-dimensional, and even five-dimensional scans may also be captured of the implanted object. These scans may also be provided to the implant identification system.

In addition to receiving the internal medical images and/or scans, the implant identification system also receives descriptive material about the implanted object that is provided by the personnel who are also providing the internal medical images, or other individuals that may have additional descriptive material regarding the implant. The internal medical images, themselves, may have descriptive material such as how the patient was positioned and possibly other specific information about the physical environment in which the internal medical image was captured. However, in addition to information about the internal medical images, the descriptive material received in step 108 describes knowledge that the personnel have about the implanted object.

The descriptive material may, for example, include a date or timeframe (e.g., the year or a cutoff year if the exact year of implantation is not known, e.g., before 1980) that the object was implanted in the patient, a geographical location (e.g., a country) in which the object was implanted in the patient, the type of material from which the implant is constructed, the type of implant (e.g., hip replacement, spine implant, stent, pacemaker, wrist replacement, elbow replacement, knee replacement, screws, plates, intramedullary nail, wire mesh, heart valve, ankle replacement, dental and maxofacial implants, or other type of medical-related implant). Descriptive information can also include partial identification of implant, identification of characteristics, identification of various components, identification of implant fixation, identification of specific primary surgeon or revision surgeon, and the facility of primary or revision surgery.

For example, the implant identification system may be implemented as a web-based service. The personnel at the remote location may use a web browser to reach a user interface page provided by the implant identification system. Via the user interface screen, the personnel may be able to attach or otherwise submit the internal medical images and the scan data and also be presented with text-boxes or drop-down menus that allow them to enter descriptive material. In particular, the available drop-down menus and text-boxes may be dynamically generated based on the personnel identifying the type of implant that the implanted object is or by the type of data that is being attached by the personnel. For example, if the implanted object is a stent, then the personnel may be presented with different options and choices for entering the descriptive material as compared to if the implanted object was a hip-replacement. Furthermore, the options and choices may be different if the personnel indicate they are attaching a CT or MRI scan in addition to or instead of the internal medical images. The implant identification system then uses the received information, in step 110, to identify the implanted object from a database of information about medical-related implants. The identification of the implanted object can be performed in distinct stages. For example, the amount of data in a two-dimensional internal medical image will likely be far less than the data in a three-dimensional scan. Thus, the implant identification system may first attempt to identify the implanted object based on the internal medical images. If identification using the internal medical images does not produce a unique result, then there may be a plurality of implants in the database that possibly match the implanted object.

The provided descriptive material may then be used to further refine the list of possible matching implants. For example, if the material from which the implanted object is constructed is provided as the descriptive material, then that may be used to eliminate choices that were returned as possible matching implants. Along these lines, any type of descriptive information may be used to refine the list of possible matching implants.

The acquisition of the descriptive material may be interactive in nature as well. For example, if the implant identification system determines that there are two possible hip replacement implants in the database that match the characteristics of the internal medical images, then the implant identification system may compare the information in the database for those two possible hip replacement implants. Based on the comparison, the implant identification system may determine that the countries of origin of the two hip replacement implants are different and that the material of their construction is different as well. Based on these distinctions, the implant identification system may send an inquiry message to the personnel asking if the personnel can provide information about where the implant surgery was performed or if they know the material from which the implant is constructed. Based on this specific inquiry, the personnel can provide particular descriptive material that allows the implant identification system to choose the correct hip replacement implant from the database as matching the implanted object.

In some instances, the implant identification system may provide a list of the possible matching implants and allow the personnel to select one or more for receiving additional information. This may be the case, for example, if the implant identification system is unable to limit the possible matching implants down to a single implant.

In step 112, based on the personnel's selection or based on the unique identification of the matching implant, the implant identification system retrieves operational characteristics about the identified matching implant. Different operational characteristics about a medical-related implant may be useful for different uses. But in general, the operational characteristics can include information such as: what type of implant the object is, the manufacturer of the object, the model of the object, whether the object is subject to a recall, what other devices would be compatible with the object that is identified, what current substitutes are available for the object, what equipment is related to implanting, revising, or replacing the object, a point-of-contact for obtaining a replacement for the object, a material composition of the object, a material texture of the object, and physical dimensions of the object, what sizes and tapers and ratios are possible (e.g., cup to liner to head to neck to stem), what heads, leads, valves, mesh and screw to plate are possible, what equipment is needed to be able to revise the implanted object, what are the allowed metallurgy combinations of other implant components, etc. One of ordinary skill will recognize that other operational characteristics may be retrieved in step 112 as well without departing from the scope of the present invention.

In step 114, the operational characteristics of the implanted object are then transmitted to the personnel that provided the internal medical images or, alternatively to other or alternate parties as well. In the web-based example provided above, the personnel may be provided with a report than can be saved or printed. Additionally, the report can be emailed, or otherwise transmitted, to the personnel or other third parties that they designate. An additional feature of the system is that it may have the ability to communicate with various electronic medical record (EMR) systems for direct download into the patient's medical record, such that one or more operational characteristics of the implant may be obtained automatically from the system. The implant identification system may also access EMR systems to upload descriptive material about the patient's implants.

The operational characteristics identified by the implant identification system may or may not go to the facility where the images were taken. For example, the images may be sent from a patient, a surgeon's phone or mobile device, a surgeon's computer, a hospital, a surgery center, an attorney's office, etc. Thus, while the images may be sent from a location where they are taken, the images can also be sent from locations that are distant from where the images were taken. Similarly, the descriptive material may be sent from a device or location from which the images were sent or can be from an entirely separate location or party.

FIG. 1B depicts a flowchart of an example method for refining a selection of matching implants in accordance with the principles of the present invention. As mentioned above, in describing step 110, the implant identification system may initially, in step 120, identify a plurality of possible matching implants based on the received internal medical images and descriptive information. If available, any three-dimensional scan information may then be used to see if any of the possible matching implants can be eliminated from the list of possible matches.

Additionally, the descriptive material that the personnel initially submit may be used, in step 122, to eliminate as many possible matching implants from the list of possible matches. As an option, in step 124, the implant identification system can request specific additional descriptive material to further reduce the number of possible matching implants.

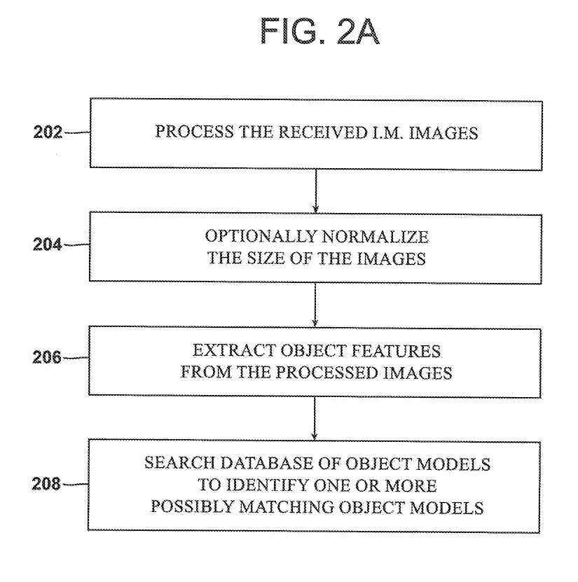

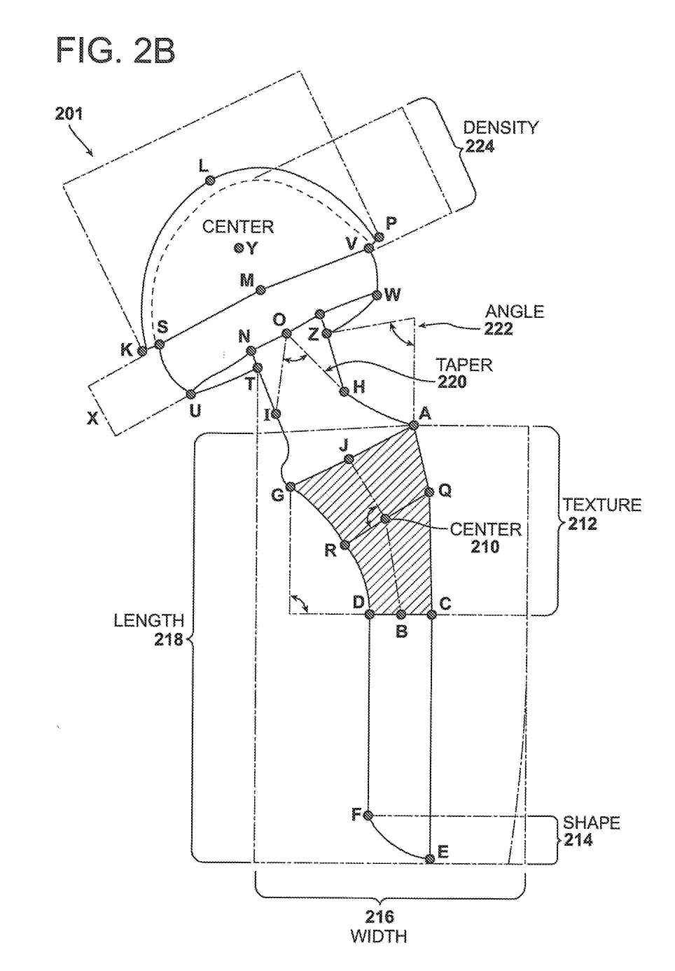

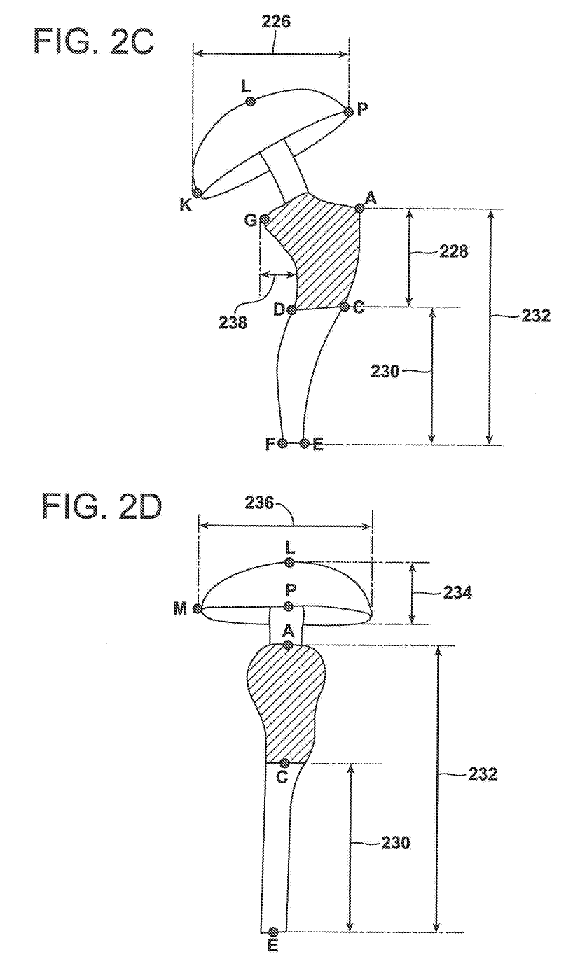

FIG. 2A depicts a flow chart of an example method for identifying matching implants in a database in accordance with the principles of the present invention. One of ordinary skill will recognize that there are a variety of different techniques to perform object recognition. Different techniques are useful when there is a priori knowledge about the internal medical images (e.g., two images are available from mutually orthogonal perspectives; or the images are gray scale images) or there is a priori knowledge about the subject of the images (e.g., the subject of the image is an anterior-posterior view of a knee replacement implant). There are also a number of known techniques for using three-dimensional data to generate two dimensional slices of an implanted object as well as using two dimensional images from different perspectives to calculate volumetric data about an implanted object. Embodiments of the present invention encompass using any of the widely varied and known techniques to recognize and identify objects in an image, such as image pattern recognition algorithms, computer-assisted detection programs, computer-assisted diagnosis programs, or content-based image retrieval software.

In step 202, the implant identification system may initially process the received internal medical images and/or any scan data. For example, the internal medical images may be filtered to sharpen edges and to remove noise and other artifacts that may obscure details about the internal medical images. The contrast and other image pixel values may be normalized or otherwise altered to generate an internal medical image in a desired format for use by the implant identification system.

In step 204, the implant identification system may normalize the size of any internal medical images that are received, e.g., the system may re-size the internal medical images to a predetermined size. For example, the internal medical image may initially be captured with a phantom object of a known length. The implant identification system will expect the presence of such a phantom and also search for the location in predetermined regions in each internal medical image. Using the presence and size of the phantom of a known length, the implant identification system can re-size the internal medical image to a predetermined resolution (e.g., 10 pixels equal 1 mm) and allow the implant identification system to calculate physical dimensions of the implanted object. One of ordinary skill will recognize that there are also other techniques that can be used to embed dimensional information in the internal medical image itself.

In step 206, the implant identification system analyzes each internal medical image to extract features (e.g., textures, lines, densities, boundaries, corners, edges, transitions, center-of-mass, angles, etc.) of the implanted object. Having at least two internal medical images available for feature extraction allows more robust information to be determined for the implanted object in the images. For example, a single two-dimensional image only provides distance in the plane of the image to be calculated but additional images allow three-dimensional distances between object features to be determined as well.

The particular object features and physical dimensions and ratios that may be extracted in step 206 can vary depending on the type of implant. For example, a spine implant may have different features of interest than an ankle implant. Accordingly, the implant identification system might rely on the personnel submitting the internal medical images to identify (at least tentatively) the implanted object. Alternatively, the implant identification system may employ an initial process that first identifies the type of implant that the implanted object likely is. Once the type of implant is identified, then the predetermined features of interest for that type of implant can be retrieved and used to control what features are extracted from the internal medical images in step 206.

The implant identification system includes a database of implant models that describe the physical features of each implant and may also include respective images of each implant from a variety of perspectives. The database can also include, for example, engineering design data and computer-aided-drafting (CAD) drawings of each implant. In general, each implant model is a description of that implant that can be used to compare to the features that are extracted from an image in step 206. Thus, in step 208, the implant identification system searches the database of implant models to identify the one (or possibly more) implant models that have dimensions or features that are similar to the implanted object of the provided internal medical images.

The search and identification of matching implant models from the database does not necessarily have to be a binary decision of match/mismatch. Instead, the similarity between an implant model and the features of the implanted object can be scored so that relative scores between different implant models can be compared.

For example a score from 0-100 can be calculated that indicates how similar the extracted features of the implanted object are to the data within an implant model. The implant identification system can then use heuristics to determine what results to provide the personnel who submitted the internal medical images or any other third party. For example, if five implant models received a score between 60 and 85, then the implant identification system may select the highest scoring model as potentially matching the implanted object. Also, for example, if no implant model has a score over 40, then the implant identification system may report that no matching implant models were found. Also, if two different implant models both receive a score over 95, then the implant identification system may report that two matches were found or may use additional descriptive material to eliminate one of the matches or additional images may be requested as described herein.

FIGS. 2B-2D depict an example hip implant 201 having example features of interest marked with circles that each have a letter label. FIG. 2B is provided merely by way of example to illustrate how different points A-Z on an implant 201 can be utilized. For example, the polygonal region bounded by points A-Q-C-B-R-G-J can be analyzed by the system in such a way to identify the material composition of this region. For example, the texture 212 of this region and/or its density may be used to help identify its composition (e.g., stainless steel, ceramic, teflon, etc.). The curve between points F-E can be analyzed by the system to identify its “shape” 214. Some of the points of interest used for one purpose can also be used for other purposes. For example, the length 218 between points A-E can be used to describe one feature (e.g., femoral hip stem length) of the hip implant 201. Similarly, the density 224 of the image region bounded by points K-L-P-M can be used to identify its material composition. In this way, the material composition of the acetabular liner along with the composition of the femoral hip neck and hip head may help identify the specific hip implant 201. A width between points K-P or between points T-A can also be calculated.

The region bounded by the points S-M-V-W-O-N-U-S may pertain to an amount (length X) that the femoral head extends below the acetabular liner. This feature of the hip implant 201 may be useful in its identification. An angle 222 between points A-Z can be extracted as can a taper 220 using points I-O-H. The distances between point U-T or Z-W may be helpful in determining characteristics about the femoral head of the hip implant 201. Additionally, a center point 210 of a region (or the entire implant 201) can be located as a point, or feature, or interest. In this way, the points of interest 210 provide a way to capture the physical dimension of implant 220 that may help to find similar implant models in the database of implant models.

In addition to the physical dimensions, ratios between various dimensions can be calculated as well using the points A-Z of FIGS. 2B-2D. For example, the two different views of the hip implant 201, as shown in FIGS. 2C and 2D illustrate example lengths (or widths) 226, 228, 230, 232, 234, 236, and 238. One of ordinary skill will recognize that a plurality of other physical dimensions could also be extracted using the points A-Z as shown in FIG. 2B. These different physical dimensions allow a number of ratios to be calculated as well. For example, the ratio between 226 and 232 may be helpful in identifying the hip implant 201.

Thus, the internal medical images can be used to calculate, identify or determine such features of the hip implant 201 as volume, contrast, size, shape, length, width, texture, angles, density, height, area, configuration, combination, position, direction, and location. The points of interest visible in one image or across multiple images can also be used, in conjunction with the above physical attributes, to create one or more ratios. These ratios and physical attributes can be used in various combinations in order to automatically identify a specific implant. One benefit of using ratios as identifying features is that these ratio values are useful even if a provided internal medical image and corresponding images of the implant identification system have different image resolutions or sizes.

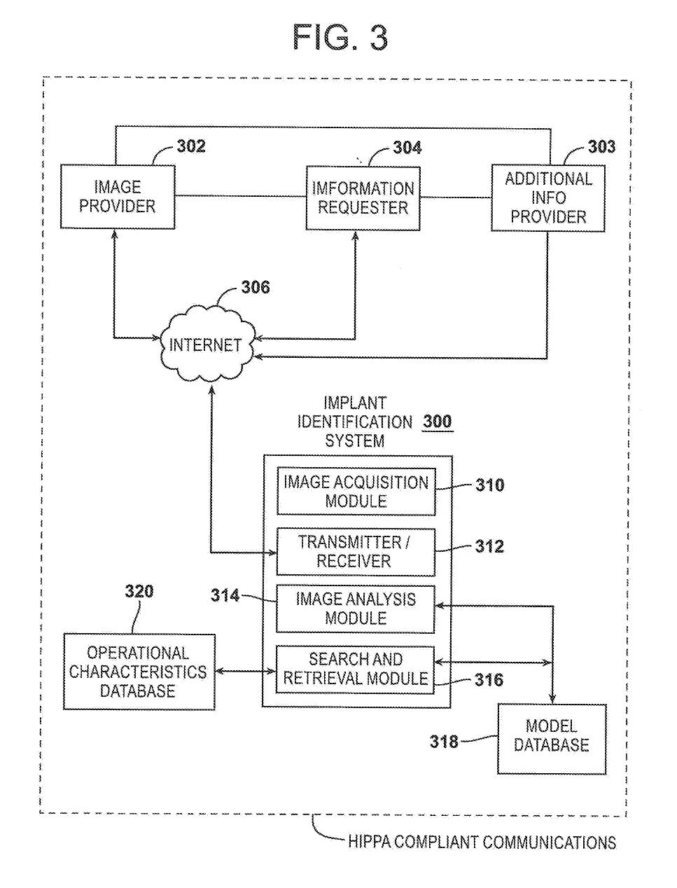

FIG. 3 depicts a system for identifying implants in accordance with the principles of the present invention. FIG. 3 provides a block-level diagram of a computer-based system that can be used to implement the methods described above with reference to FIG. 1A, FIG. 1B, and FIG. 2A. As such, some of the details described before are not fully described again. However, the messages and their contents and information that are exchanged between parties and that may be included in the various databases are intended to encompass the broad range of information described earlier. As shown by the dotted outline around the communicating parties, each communication may be performed in such a way as to comply with all Health Insurance Portability and Accountability Act of 1996 (HIPPA) requirements or those of similar privacy-related regulations.

An image provider 302 and an information requester 304 are shown as separate entities in FIG. 3. However, these two entities may also be the same as was described with reference to FIG. 1A. Regardless of whether or not they are separate entities, the image provider 302 may communicate with the implant identification system 300 to provide internal medical images, possibly scan data, and other descriptive material about an implanted object. The information requestor 304 or another information provider 303 may also provide additional images and other descriptive material about the implanted object. In addition, only a single, respective image provider 302, information requester 304, and additional information provider 303 are shown in FIG. 3. However, more than one image provider, information requester, or additional information provider can exist at different locations without departing from the scope of the present invention.

The image provider 302 may be located remotely from the implant identification system 300 and communicate using the internet 306 or other network. The implant identification system 300 includes a transmitter/receiver 312 that can receive information from the internet 306 and also transmit information over the internet 306 as well. For example, the implant identification system 300 can include a web server front-end that receives information from a web browser and provides information in a format to be displayed on a web browser.

In particular, the transmitter/receiver 312 can receive internal medical image information and possibly three-dimensional scan information that can be passed to an image acquisition module 310. The image acquisition module 310 may communicate with an image analysis module 314. In particular, the internal medical images acquired by the image acquisition module 310 can be analyzed by the image analysis module 314 to extract features and physical attributes related to the implanted object that is the subject of the acquired internal medical images.

An implant model database 318, as described earlier, can include data and images that describe a particular medical-related implant. The model database 318 can include such information for a plurality of different implants and implant types. This information can be collected from implant suppliers and historical data that is available in paper and electronic form. More particularly, as much physical dimension information, images and drawings are collected in the model database 318 that will allow comparison of images of implanted objects with the database 318 in order to identify possible matching implant models. The search and retrieval module 316 works in conjunction with the image analysis module 314 to identify which implant model(s) in the model database 318 matches the implanted object in internal medical images provided by the image provider 302 or information requester 304.

In addition to the information in the model database 318 that allows identification of matching implant models, the implant identification system 300 also includes an operational characteristics database 320 with respective operational characteristics for each of the implant models in the model database 318. The operational characteristics database 320 includes additional information, as described earlier, which may be useful for medical, legal, or criminal reasons in relationship to the patient or the implanted object in the patient. The search and retrieval module 316 retrieves the operational characteristics of the matching implant model from the database 320 and forwards that to the image provider personnel 302 or an information requester 304.

The implant identification system may be implemented as a web based business method, for example. The personnel providing the internal medical images may be a one-time user that can be charged on a per-use basis, but more realistically, these personnel can be subscribers to an implant identification service and be charged on a subscription basis or a combination of subscription plus per-use basis.

The implant identification service builds the initial implant model database and operational characteristics database utilizing available books, industry information, electrical records, and subject-matter experts. Access to these databases provides value for which the implant identification service can charge a subscription fee. The personnel providing the internal medical images are provided login permission to the implant identification service and can submit internal medical images, scan information, and additional descriptive material. In return, the implant identification service will identify a possibly matching implant from their databases and also provide operational characteristic information to the internal medical image provider about the implanted object. The implant identification service can (in a way that protects patient privacy) also collect the information submitted by various internal medical image providers and optionally augment their databases with this additional information. Additionally, internal medical image providers may be able to upload implant information and be rewarded with a reduced subscription fee.

While any of a wide variety of additional descriptive material can be sent to the implant identification system to assist in identifying implants, some specific examples discussed above include: location of primary surgery, location of revision surgery, year or date range of surgery, cemented/non-cemented, modular, non-modular, articulation surface, coating, texture, taper, neck angle, size, manufacturer, additional manufacturers, type of implant, location of implant, material that the implant is made from, surgeon who implanted the primary implant, surgeon who implanted the revision implant, year of the patient's birth, name or location of the medical facility where the implant was implanted, the manufacturer of other implants in the patient, other known implants in the patient, (e.g., from the contra-lateral side), and bearings surface.

In addition, the operational characteristics which the implant identification provides can include a wide variety of information as well. Some examples discussed above include: what type of implant the object is, the manufacturer of the object, the model of the object, whether the object is subject to a recall, what other devices would be compatible with the object that is identified, what current substitutes are available for the object, what equipment is related to implanting, revising, or replacing the object, a point-of-contact for obtaining a replacement for the object, a material composition of the object, a material texture of the object, and physical dimensions of the object, surgical technique for that implant, consulting options to discuss that implant, and places to refer that implant. Additional example information can include: the year of manufacture of the implant, the bearing services available, marketing information related to the implant of substitute implants, educational opportunities related to the implant or substitute implants, taper, metallurgy, compatibility, and registry data related to the implant.

Some benefits of the implant identification system described above may include reduced operating room time, reduced surgical time, reduced time to surgery, reduced time to implant identification, reduction in incorrect equipment and implants in the operating room, reduced infections due to reduced operating room time and the amount of implants being implanted in the patient, reduced cost to the healthcare system, reduced number of implants needed in revision, shorter lengths of stay for a patient in the medical facility, decreased patient morbidity, reduced patient mortality, better alignment and outcomes due to proper equipment and instruments in surgery, decreased resources needed to identify an implant, and decreased amount of time needed to identify implants.

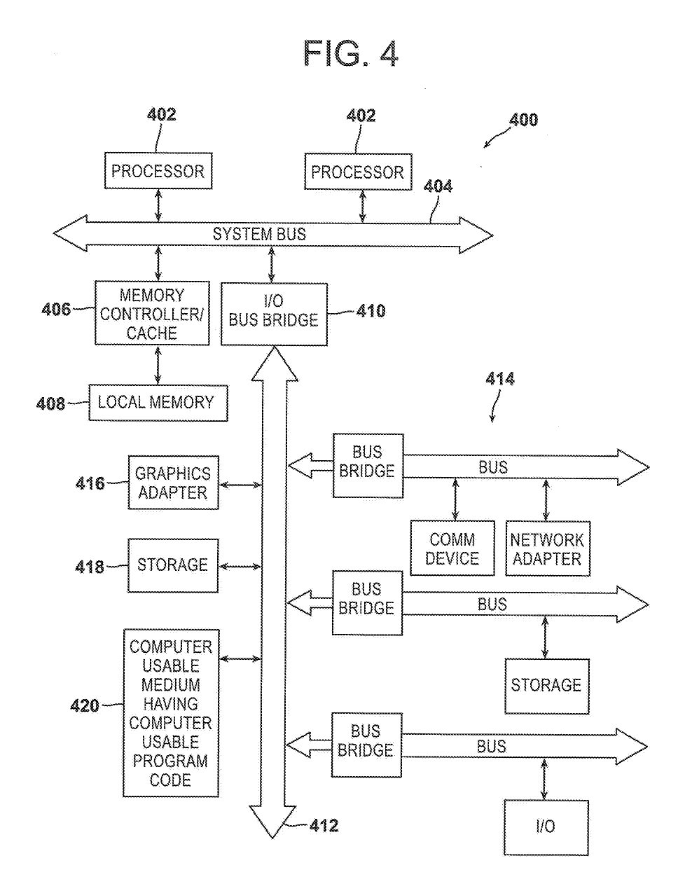

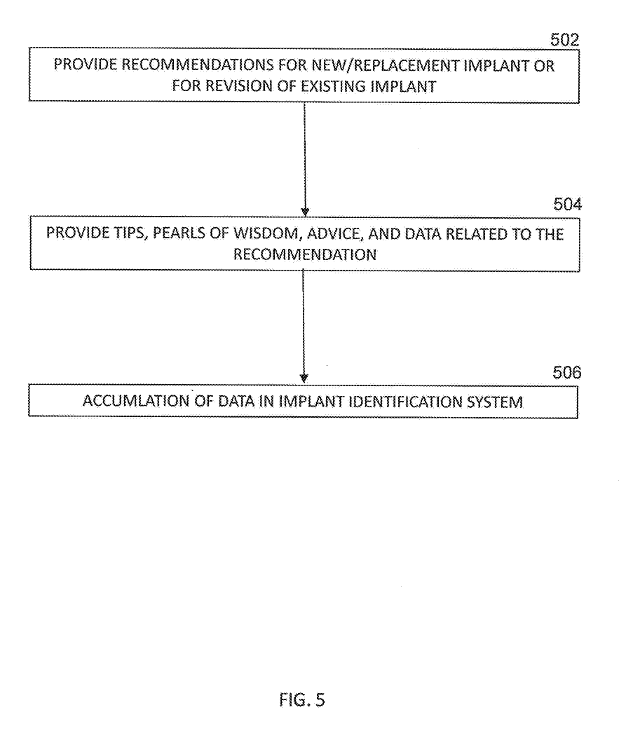

Referring to FIG. 4, a block diagram of a data processing system is depicted in accordance with the present disclosure. A data processing system 400, such as may be utilized to implement the implant identification system 300 or aspects thereof, e.g., as set out in greater detail in FIG. 1A-FIG. 3, may comprise a plurality of processors 402 connected to system bus 404. Alternatively, a single processor 402 may be employed. Also connected to system bus 404 is memory controller/cache 406, which provides an interface to local memory 408. An I/O bridge 410 is connected to the system bus 404 and provides an interface to an I/O bus 412. The I/O bus may be utilized to support one or more busses and corresponding devices 414, such as bus bridges, input output devices (I/O devices), storage, network adapters, etc. Network adapters may also be coupled to the system to enable the data processing system to become coupled to other data processing systems or remote printers or storage devices through intervening private or public networks.

Also connected to the I/O bus may be devices such as a graphics adapter 416, storage 418 and a computer usable storage medium 420 having computer usable program code embodied thereon. The computer usable program code may be executed to execute any aspect of the present disclosure, for example, to implement aspects of any of the methods, computer program products and/or system components illustrated in FIG. 1A-FIG. 3, FIGS. 5-7.

Aspects of the present disclosure may be implemented entirely via hardware, entirely via software (including firmware, resident software, micro-code, etc.) or by combining software and hardware implementation that may all generally be referred to herein as a “circuit,” “module,” “component,” or “system.” Furthermore, aspects of the present disclosure may take the form of a computer program product embodied in one or more computer readable media having computer readable program code embodied thereon.

Any combination of one or more computer readable media may be utilized. The computer readable media may be a computer readable signal medium or a computer readable storage medium. A computer readable storage medium may be, for example, but not limited to, an electronic, magnetic, optical, electromagnetic, or semiconductor system, apparatus, or device, or any suitable combination of the foregoing. More specific examples (a non-exhaustive list) of the computer readable storage medium would include the following: a portable computer diskette, a hard disk, a random-access memory (RAM), a read-only memory (ROM), an erasable programmable read-only memory (EPROM or Flash memory), an appropriate optical fiber with a repeater, a magnetic storage device, or any suitable combination of the foregoing. In the context of this document, a computer readable storage medium may be any tangible medium that can contain, or store a program for use by or in connection with an instruction execution system, apparatus, or device.

In contrast, a computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device. Program code embodied on a computer readable signal medium may be transmitted using any appropriate medium, including but not limited to wireless, wireline, optical fiber cable, RF, etc., or any suitable combination of the foregoing.

Computer program code for carrying out operations for aspects of the present disclosure may be written in any combination of one or more programming languages, including an object oriented programming language, conventional procedural programming languages, dynamic programming languages or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (SaaS).

Aspects of the present disclosure are described herein with reference to flowchart illustrations and/or block diagrams of methods, apparatuses (systems) and computer program products according to embodiments of the disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable instruction execution apparatus, create a mechanism for implementing the functions/acts specified in the flowchart and/or block diagram block or blocks.

These computer program instructions may also be stored in a computer readable medium that when executed can direct a computer, other programmable data processing apparatus, or other devices to function in a particular manner, such that the instructions when stored in the computer readable medium produce an article of manufacture including instructions which when executed, cause a computer to implement the function/act specified in the flowchart and/or block diagram block or blocks. The computer program instructions may also be loaded onto a computer, other programmable instruction execution apparatus, or other devices to cause a series of operational steps to be performed on the computer, other programmable apparatuses or other devices to produce a computer implemented process such that the instructions which execute on the computer or other programmable apparatus provide processes for implementing the functions/acts specified in the flowchart and/or block diagram block or blocks.

FIG. 5 depicts a flow chart of an example method for recommending one or more potential implants or revision techniques to a user in accordance with the principles of the present invention. The implant identification system described herein may be used for the recommendation, or a separate recommendation engine, which may or may not communicate with the implant identification system, may be used for the recommendation. In the present example, the implant identification system is used for the recommendation. Thus, one or both of the model database 318 or the operational characteristics database 320 can be augmented to include additional information, as discussed below, useful for providing recommendations. One of ordinary skill will recognize that the databases do not have to store all available information locally but can include links or pointers to content stored geographically remote from the implant identification system 300. Furthermore, as mentioned above, the recommendation engine, and any accompanying data storage, may be separate from the implant identification system 300.

In step 502, the implant identification system may provide one or more recommendations for a new or replacement implant to be inserted into a patient, or for revision techniques that could be used by a surgeon, for example, by transmitting the recommendations to the user of the implant identification system. The implant identification system may advantageously use the identified implant information determined from the methods and processes set out herein in determining the replacement implant(s) or techniques, or the implant identification system may use other sources of information. For example, the recommendation(s) may be based off of predetermined settings in the system as will be described in greater detail below.

At step 504, the implant identification system can also provide the user with tips, pearls of wisdom, advice, and data related to the recommendation, for example, by transmitting the information to the user. The information provided to the user can be based on the previously identified implant(s), which information may have been flagged in the implant identification system or by other sources as being important. The more the implant identification system is used, the more contributing comments, information, and advice can be shared between users.

The recommendations and data may be based on any of the following: implants, products, equipment, processes and protocols, and/or techniques that could potentially be used or have been used before in a similar situation.

As one example of this aspect of the invention, after the implant is identified as set out herein, the implant identification system recommends one or more potential replacement implants or techniques, equipment to be used in the procedure, or protocols that could be used by the surgeon in revising, changing, augmenting or explanting the implant. The implant identification system may or may not ask for additional information from the user prior to offering these recommendations.

At step 506, the implant identification system accumulates information as it is used, i.e., the implant identification system gathers implant information and recommendation information as it is used and as users select recommended implants or techniques based on recommendations from the implant identification system. Such information accumulation facilitates more automation and more accurate recommendations based on prior inputs, thus creating a more artificially-intelligent system.

As noted above, the recommendations made by the implant identification system according to this aspect of the invention are not limited to potential new/replacement implants, as they could also relate to techniques to be used on an existing implant. Such techniques may include, for example, surgical approaches, surgical solutions, surgical processes, surgical plans, surgical hypotheticals, e.g., “if X, Y, and/or Z occur during a procedure, then take steps A, B, and/or C”, material, or sizes and shapes. The implant identification system may provide such recommendations or information to the user based on one or more of the following:
1.) Additional information provided by the user, e.g., wherein the user answers more questions, supplies more data or uploads more images;
2.) Current information that is determined from analysis of data already provided, such as internal medical images, implants, osteolysis, implant position, implant wear, etc.;
3.) Prior case information that has been accumulated via use of the implant identification system, e.g., as discussed in step 506; and/or
4.) Settings or preferences applied to the implant identification system by itself, a user, or a third party. For example, the user can choose a company and then the recommendation system will recommend a product from that company to be used in revising the current implant. The system can also provide the user a surgical plan incorporating specific products as well as equipment. The system may also offer videos and techniques to the user based on that company and implant.

In a possible example of this technology, a company may pay for its product or solution to be offered to the user so the user will see those recommendations/options when using the implant identification system. The system in turn recommends to that company, a particular system, and one or more possible surgical approaches to be used.

In another possible example, the user may choose a company, wherein the implant identification system will then recommend a product from that company to be used in revising the current implant or in recommending a new or replacement implant. The system may also provide the user with a surgical plan, which may incorporate specific products and/or equipment. The system may also offer instructional videos and techniques to the user based on that company and implant. The system may further offer the user the opportunity to speak to an expert on that product or surgical procedure.

According to this aspect of the invention, the recommendations from the implant identification system can be provided manually or automatically, and can be provided in real-time or within a period of time. In one example, the implant recommendation may be generated by the implant identification system analyzing the images and additional information provided by the user, where the implant identification system may then optionally, if required, ask the user or another third party for additional information. The implant identification system then uses the information to make a recommendation. The recommendation may optionally be (at least partially) based on participating companies that paid for the service, as discussed herein.

A primary benefit of this aspect of the invention is that, after the identification of the implant by the implant identification system, the implant identification system can then help the user in the next steps. In a working example of this technology, a surgeon may desire information on his/her options for revising the identified implant. Based on data the implant identification system has (or can acquire as set out herein), the implant identification system can offer recommendations to the user. In one example, this recommendation may include equipment needed for a revision of the identified implant. In another example, the recommendation may include equipment needed for removing the identified implant and then inserting a replacement into the patient, along with recommendations for the replacement implant including surgical approaches that can be used for the procedures. Each of these recommendations offers an opportunity for a company to place their products or services into the pool of possible recommendations.

While companies that manufacture implants may recommend their branded implants for implementation into the patient, the implant identification system may also be used by companies that manufacture other types of products that may be used in a surgical implantation procedure. Such products include but are not limited to, for example, surgical beds, positioners, tables, clamps, retractors, sutures, lights, inter-op meds, inter-op pain meds, power equipment, and other types of medical devices, e.g., cutting apparatuses, drills, drapes, cement, etc.

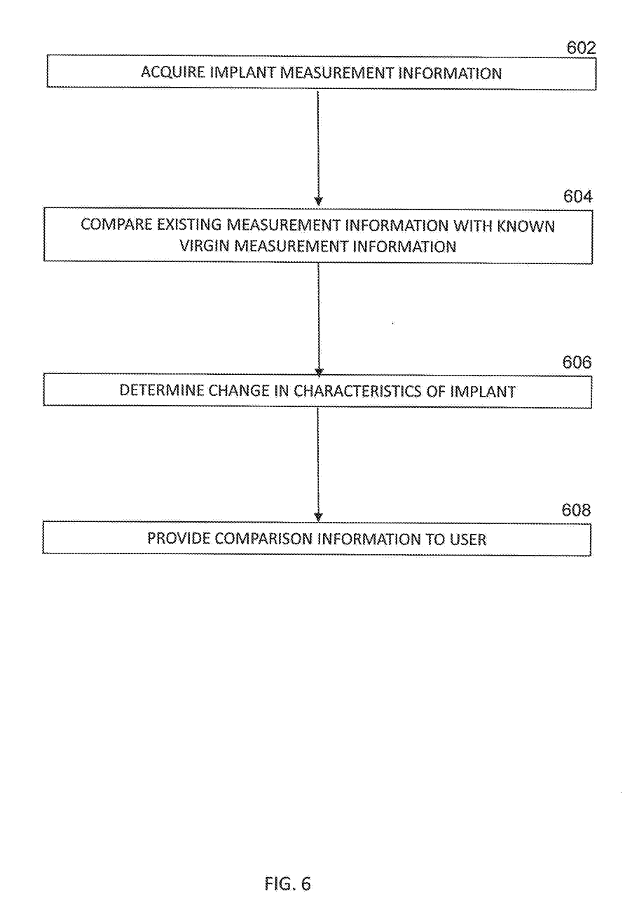

FIG. 6 depicts a flow chart of an example method for estimating wear of an implant in accordance with the principles of the present invention. The implant identification system described herein may be used for this process, or a separate estimation engine, which may or may not communicate with the implant identification system, may be used for this process. In the present example, the implant identification system is used.

In step 602, the implant identification system acquires measurement information of the existing implant (hereinafter “existing measurement information”). This measurement information may be acquired internally from memory, e.g., based on the images supplied by the user as set out herein, or from an external source.

At step 604, the implant identification system compares the existing measurement information with known virgin measurement information pertaining to the implant (hereinafter “virgin measurement information”) to determine comparison information, wherein the virgin measurement information may be based on a new, unused version of the implant.

Based on the comparison, at step 606 the implant identification system determines a change in the characteristics, e.g., size, shape, definition (corner and edge sharpness), etc., of the implant. This can be accomplished with mathematical algorithms or based purely on a comparison of images of the existing implant and images of a virgin version of the implant.

At step 608, the implant identification system provides the determined comparison information to the user, for example, by transmitting the information to the user. This information may be used by the user, a surgeon, healthcare provider, etc., to determine the amount of wear an implant has sustained over a period of time. While variables could obviously have a significant impact on the life of an implant, the user/surgeon/provider could also use this information to predict the remaining life span of the implant. Such a remaining life span prediction could be used to help the surgeon and/or patient decide whether a revision to the existing implant or a replacement implant is warranted.

A primary goal of this aspect of the invention is to estimate the wear sustained by an implant over time, thus ultimately facilitating better care for the patient. This aspect of the invention could also be used to identify excessive wear patterns, which may lead to other problems such as osteolysis. This information would not only be helpful to healthcare providers, it could also be useful to implant manufacturers, in that it provides an opportunity to monitor how implants are performing over time. If an implant has shown excessive wear, or if it is found that a particular implant is consistently experiencing wear in a specific area or areas, it may be a sign of issues with the implant that could then be addressed by the manufacturer.

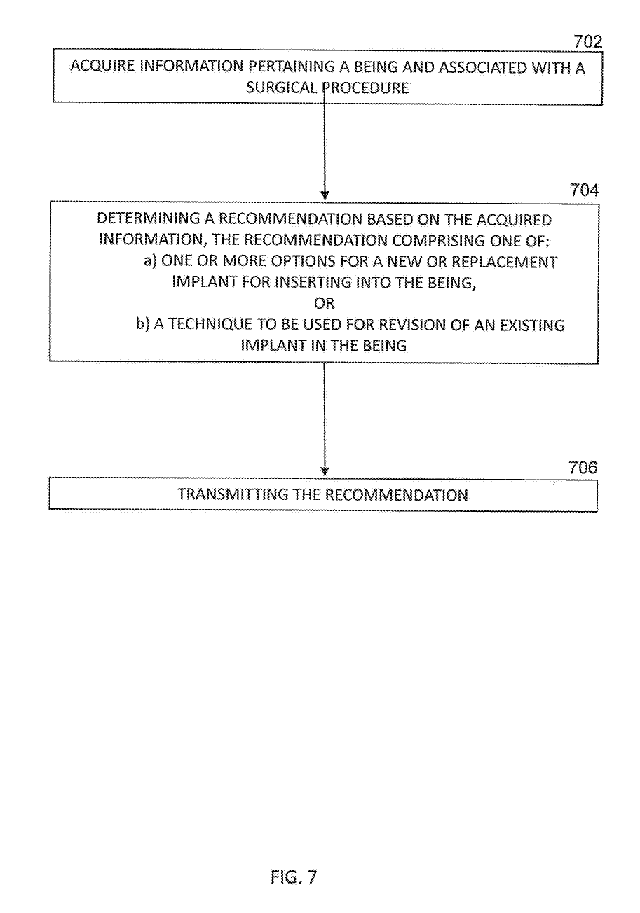

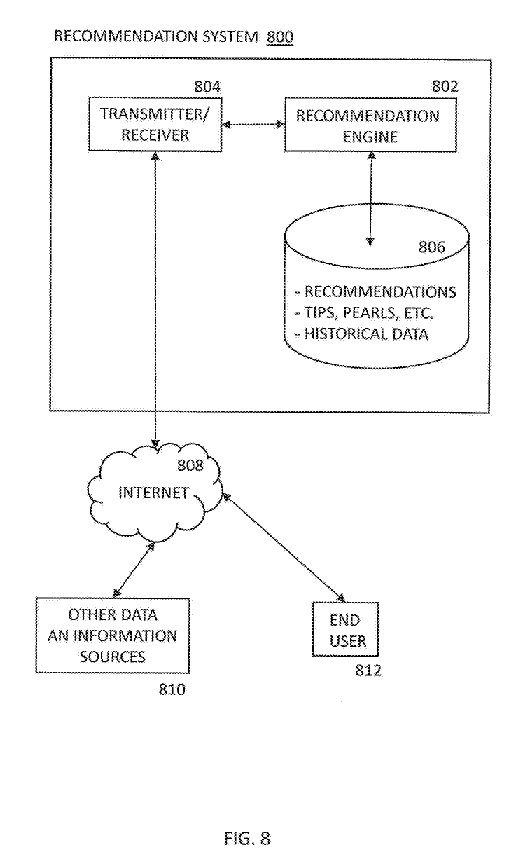

FIG. 7 depicts a flowchart of an example method, from the perspective of an implant recommendation system, for providing a recommendation in accordance with the principles of the present invention. FIG. 8 depicts a block-level diagram of an implant recommendation system in accordance with the principles of the present invention. Unlike the embodiment described with respect to FIGS. 5 and 6, the implant recommendation system 800 of FIG. 8 is separate from the implant identification system 300, although the implant recommendation system 800 of FIG. 8 could be a part of the implant identification system 300.

Although the examples described herein relate to an implant already existing within a patient, the present recommendation engine can be used as a tool for revision surgery or primary surgery without departing from the scope of the present invention.

Returning to FIG. 7, the implant recommendation system, or the recommendation engine, can, in step 702, acquire information pertaining to a patient (i.e., a being) and associated with a surgical procedure which is being performed or will be performed in the future. In terms of FIG. 7, the “user” is a person or system to which the recommendation and any accompanying information is being provided. As discussed above, the recommendation engine can be used to provide the user options as to what surgical approach, equipment, instruments, implants, or protocols to use when revising, changing, or augmenting an implant that may or may not have been identified using the implant identification system 300.

Of the many possible or available recommendations that could potentially be provided to the user, the recommendations that are ultimately provided can be limited or filtered automatically or manually. “Automatically” means there are pre-set recommendations based off preferences set in a user profile stored by the recommendation system and “manually” means the provided recommendations will be based on data provided by the user at the time they use the recommendation engine.

One of ordinary skill will recognize that the information acquired by the recommendation engine in step 702 can vary without departing from the scope of the present invention. Furthermore, the more information that can be acquired may be beneficial in identifying a specific recommendation from among all the available alternatives. Thus, the recommendation engine may require a minimum amount of information be acquired before a recommendation can be made.

By way of example, the recommendation engine may require a user to provide at a minimum both a) and b), as described below, so that the recommendation(s) provided to the user can be based on one or more of these factors.

a) at least 2 of the following: the pathology related to the case, intended surgical approach for the case, age or activity level of the patient, or reason for the surgery; and

b) any one (or more) of the following:
i) the images or implants that have been identified by the implant identification system;
ii) predetermined settings in the system (settings which are set by the customer, user, patient, manufacturer or 3rd party, for example, an insurance company or hospital);
iii) the current implants in the patient identified by the user;
iv) the current implants in the patient identified by other means;
v) equipment and implant compatibility (e.g., does a hip stem have a 12/14 taper and if so what heads have the same 12/14 taper and in-turn what acetabular cups and liners can be implanted and used with that head);
vi) equipment and implant availability (e.g., a product may have been recalled and not available, may have been pulled from the market for any number of reasons, may not be manufactured any longer, etc.);
vii) equipment, implant and surgical approach indication;
viii) equipment, implant and surgical approach contra-indication;
ix) the price of implants;
x) the available medical plan or insurance plan;
xi) the insurance accepted by the physician or facility;
xii) the number of prior surgeries of the patient;
xiii) a user-specified preference;
xiv) a drop down menu of options allowed by that patient's insurance plan;
xv) a drop down menu of options allowed by hospital or surgery center contracts;
xvi) inputs by the provider for that specific case;
xvii) the patient's paper or electronic medical records (e.g., if the medical records say the patient has a DePuy Sigma knee size 4 femur, size 4 tibia and 10 mm thick poly. The recommendation engine may offer a few options of implants that equal size 4 but with a more stabilized insert to achieve that goal);
xviii) templating for that particular case; and
xix) other users of the system approved to provide feedback and advice to another user.

The method of FIG. 7 continues in step 704 with the recommendation engine determining a recommendation based, at least in part, on the information acquired in step 702. In particular, the recommendation that is determined is at least one of a) one or more options for a new or replacement implant for inserting in the patient, or b) a technique to be used for revision of an existing implant in the patient. For example, the recommendation may pertain to potential implants, equipment, approach or protocols that could be used by a surgeon in revising, changing, augmenting or explanting that device. As mentioned above, the recommendation system, or recommendation engine, may also provide the user with tips, pearls of wisdom, advice, videos and data related to any recommended implant, equipment and procedure. Plus, the more the system is used the more contributing comments, data, information, and feedback can be collected, stored, and shared between users and the better the system will be able to choose recommended solutions for future patients.

In practice, the recommendation engine can search through the stored information about implants, procedures, protocols, costs, insurance plans, etc. to determine those options that match the criteria acquired from the user in step 702. Some options may match more of the user-specified criteria than other options and, thus, the available options may be presented to the user in a ranked list, or display, with the “better” options listed first. The recommendation engine can then receive input from the user that specifies additional filtering criteria. For example, if the original criteria from the user did not specify an implant size, then additional criteria can be acquired from the user that filters the options to only those having a particular implant size. As described above, other additional criteria such as insurance rules, implant costs, manufacturer, and others can be used to filter what options are considered matching options when the recommendation determines the recommendation.

Ultimately in step 706, the recommendation is transmitted or provided to the user. Below are example recommendations that may be provided in accordance with the principles of the present invention. These are merely examples and less information, or more information, could be provided without departing from the scope of the present invention. The information transmitted to the user may, for example, be provided by an interactive user-interface in which menus and pull-down items are provided such that screens of information may be provided along with the ability to drill-down to additional information or follow links to additional information or resources associated with the recommendation.

### Example Recommendation 1:

If the patient needs a revision stem through a posterior approach for a hip replacement and there are 8 different stems on contract at that hospital, the recommendation engine can offer the recommendation to the user to look at using one of those stem options.

### User Input

Type of surgery - - - Hip Replacement
Approach - - - Posterior Approach
Patient activity level - - - Low Demand
Hip Implant Stem Type - - - Wagner Style Stem

At this point the recommendation system can automatically recommend a product to use or the user can be provided with a number of different options to choose from. Thus, additional user-supplied information can be solicited (either automatically or manually) in order to filter or limit the different available options. For example, the options may be limited to companies on contract that have Wagner-style stems:

Zimmer - X Stem, Y Stem
Biomet - X Stem, Y Stem
DePuy - X Stem, Reclaim stem
S&N - X Stem, Y Stem

From these available options, there can be user preference set by the user, hospital, 3^{rd} party, or the implant identification system to additionally limit options to only Zimmer and DePuy. Further, based on the identified Zimmer and DePuy options, the recommendation system may recommend, for example, based on previously collected feedback, the DePuy stem. Thus, the user can ultimately be provided with the following recommendation:
Company: DePuy
Stem: Reclaim Stem
Head: Metal Head
Liner: Poly Liner
Cup: Sector Cup
Designed: 2010
Material: X %, Y %, Z %
Number of implants: 4 with Head

As mentioned above, additional information accompanying the recommendation may include product images, exploded-view diagrams, implant specifications, sizing information, or equipment and/or instrumentation information. Such information can include how many trays and what trays of equipment are needed, implants and what banks of implants are available and needed, what other items are needed to be at the surgery for the case to be completed, and what items are good to have at the surgery, just in case certain anticipated or unanticipated circumstances arise.

### Example Recommendation 2:

Based on the acquired information about the patient and pertaining to the surgical procedure, the recommendation system may provide the following example recommendation. As noted above, the information can be acquired from a number of different sources without departing from the scope of the present invention. For example, the following recommendations can be based, at least in part, on the acquired information being any of a) the implant identification system identifying the current implant size, b) the patient's records identifying the current implant size, c) user input that identifies the current implant size, or d) advice received from another user with experience relevant to the current procedure. The recommendation system can recommend:
Stem: Size 220 mm-240 mm
Fixation Option: Press-fit Only
Head: Metal Size 32
Insert: Poly Size 32ID and 600D Xlinked Poly
Cup: Revision Multi Hole Size 60 Gription
Screw Options: 12 Holes Ti Screws Size 10 mm-60 mm
Equipment Trays Needed: 6 Trays and what each tray has
Implant Banks Needed: 2 banks in case an implant is dropped, of sizes a-z or type X.
Retractor Trays Needed: X, Y
Ancillary Products needed: A, B, C, D, E, F
Price Est. of case: $20,000
Surgical Techniques: x, y
Videos: E, F, G
Speak to another Surgeon: Y or N
Other Tips and Pearls: H, I, J, K

In addition to the above information, the user interface of the recommendation system may include additional information such as a recommended approach. For example, a graphical user interface can be provided that displays a table with the following available options for revisions along with the recommendation.

| | Product | Recommended | | Sizes |
|---|---|---|---|---|
| Approach | DAA or Post | | | |
| Implant | DePuy \| Zimmer | DePuy | | |
| Company | | | | |
| Stem | AML\|CTP\|KRN | 15 | mm | 10-20 mm |
| Cup | Multi-Hole Pinnacle | 60 | mm | 52-80 mm |
| Liner | Poly | 36/60 | mm | 50-80 mm |
| Screws | Ti | 2-35 | mm | 6.5 mm Length |
| | | | | 10 mm-60 mm |

| | | | |
|---|---|---|---|
| Bed | Stryker \| OSI | Yes | Standard Break |
| | | | Bed with Pos |
| Retractors | Innomed | Yes | Innomed Poste- |
| | | | rior Retractors |
| Power | Synthes | Yes | Synthes Large |
| Equipment | | | Bone Power |
| Wound Closure | Ethicon | Yes | Prineo |
| Cable System | Synthes | Yes | 2 mm-5 mm |

Additional Recommendations Based on Prior Cases:
We also recommend x, y, z in addition to ABC in case of TBD

In addition to the display of the above table, or a similar table, the graphical user interface of the recommendation system can include buttons, links, or controls to allow the user access to additional information. For example, these controls can allow a user to access videos related to the surgery, request a consultation with a surgeon with experience relevant to the recommendation, browse other cases similar to the recommendation, and access information about surgical techniques involved in the recommendation.

This recommendation can then be used by the surgeon for planning the surgery. In addition, the recommendation system's user interface can include a button, or control, that can be selected by the user so that this information and recommendation can also be sent to the hospital prior to the case to make sure all of the needed equipment and instruments are ordered, available and ready for surgery. Additionally, following the procedure, the graphical user interface can be used to submit films, for example, or other feedback with specific additional information to help other surgeons in a similar situation.

Referring to the system of FIG. 8, an example recommendation system can include a recommendation engine 802 that can be embodied as a computer similar to that of FIG. 4. Thus, FIG. 8 provides a block-level diagram of a computer-based system that can be used to implement the methods described above with reference to FIGS. 5 and 7. As such, some of the details described before are not fully described again. However, the messages and their contents and information that are exchanged between parties and that may be included in the various databases are intended to encompass the broad range of information described earlier. Each communication may be performed in such a way as to comply with all Health Insurance Portability and Accountability Act of 1996 (HIPPA) requirements or those of similar privacy-related regulations.

The recommendation system 800 includes a transmitter/receiver 804 that allows the system to send information to other locations and devices and that allows the recommendation system 800 to receive information from remotely located devices and locations. In particular, the recommendations described above that are generated by the recommendation engine 802 can be sent to an end user 812. As already explained, the recommendations can be sent as part of a graphical user interface screen that includes recommendations as well as additional information. Some of the additional information may be accessible through other data sources 810. One example additional data source 810 may include the implant identification system 300 described earlier. The various systems described herein may be interconnected over a network such as the Internet 808. In the example recommendation system 800 of FIG. 8, the recommendation engine 802 is in communication with one or more databases 806 that can include the information useful for making recommendations as well as the additional tips and other information related to recommendations. Furthermore, as explained above, user interaction with the recommendation system can be monitored and additional feedback can be collected from user so that information can be accumulated about various recommendations and the pre-op and post-op circumstances surrounding their implementation by a surgeon.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A computer-implemented method for making a recommendation concerning a surgical procedure in a being, the computer including a computer readable memory storing executable instructions and a processor in communication with the computer readable memory, the method comprising:
acquiring, by the computer, information pertaining to the being;
determining, by the computer, the recommendation concerning the surgical procedure in the being using the acquired information, the recommendation comprising one of:
one or more options for a new or replacement implant for inserting into the being; or
a technique to be used for revision of an existing implant in the being; and
transmitting, by the computer, the recommendation.

2. The computer-implemented method of claim 1, wherein the recommendation comprises equipment to be used in the surgical procedure.

3. The computer-implemented method of claim 1, wherein the surgical procedure comprises one of revising, changing, augmenting, or explanting the existing implant.

4. The computer-implemented method of claim 1, wherein the information pertaining to the being comprises an identification of the existing implant.

5. The computer-implemented method of claim 4, wherein acquiring the information pertaining to the being comprises one of:
receiving the identification of the existing implant from an automated implant identification system,
receiving the identification of the existing implant from a medical record of the being, or
receiving the identification of the existing implant from user input received by a user in communication with the computer.

6. The computer-implemented method of claim 1, further comprising:
presenting, by the computer, a plurality of available options related to the surgical procedure to a user in communication with the computer;
eliminating, by the computer, one or more of the options based on input from the user to generate a set of remaining options; and
determining the recommendation from among the set of remaining options.

7. The computer-implemented method of claim 1, wherein determining the recommendation comprises:
presenting, by the computer, a plurality of recommendations to a user in communication with the computer;
receiving, by the computer, input from the user selecting one of the plurality of recommendations; and
determining the recommendation based on the selected one of the plurality of recommendations.

8. The computer-implemented method of claim 7, further comprising:
collecting, by the computer, information about the plurality of recommendations and the selected one of the plurality of recommendations.

9. The computer-implemented method of claim 8, further comprising:
using, by the computer, the collected information when determining future recommendations for other surgical procedures similar to the surgical procedure.

10. The computer-implemented method of claim 1, further comprising:
detecting, by the computer, whether the acquired information satisfies a predetermined threshold for determining a recommendation; and
before determining the recommendation, acquiring, by the computer, additional information that satisfies the predetermined threshold.

11. A system for making a recommendation concerning a surgical procedure in a being, comprising:
a memory device storing executable instructions; and
a processor in communication with the memory devices, wherein the executable instructions, when executed by the processor, causes the processor to:
acquire information pertaining to the being;
determine a recommendation concerning the surgical procedure in the being using the acquired information, the recommendation comprising one of:
one or more options for a new or replacement implant for inserting into the being; or
a technique to be used for revision of an existing implant in the being; and
transmit the recommendation.

12. The system of claim 11, wherein the recommendation comprises equipment to be used in the surgical procedure.

13. The system of claim 11, wherein the surgical procedure comprises one of revising, changing, augmenting, or explanting the existing implant.

14. The system of claim 11, wherein the information pertaining to the being comprises an identification of the existing implant.

15. The system of claim 14, wherein the executable instructions, when executed by the processor, causes the processor to:
receive the identification of the existing implant from an automated implant identification system,
receive the identification of the existing implant from a medical record of the being, or
receive the identification of the existing implant from user input received by a user in communication with the system.

16. The system of claim 11, wherein the executable instructions, when executed by the processor, causes the processor to:
present a plurality of available options related to the surgical procedure to a user in communication with the system;
eliminate one or more of the options based on input from the user to generate a set of remaining options; and
determine the recommendation from among the set of remaining options.

17. The system of claim 11, wherein the executable instructions, when executed by the processor, causes the processor to:
present a plurality of recommendations to a user in communication with the system;
receive input from the user selecting one of the plurality of recommendations; and
determine the recommendation based on the selected one of the plurality of recommendations.

18. The system of claim 17, wherein the executable instructions, when executed by the processor, causes the processor to:
collect information about the plurality of recommendations and the selected one of the plurality of recommendations.

19. The system of claim 18, wherein the executable instructions, when executed by the processor, causes the processor to:
use the collected information when determining future recommendations for other surgical procedures similar to the surgical procedure.

20. The system of claim 11, wherein the executable instructions, when executed by the processor, causes the processor to:
detect whether the acquired information satisfies a predetermined threshold for determining a recommendation; and
before determining the recommendation, acquire additional information that satisfies the predetermined threshold.
